(19) United States
(12) Patent Application Publication
(10) Pub. No.: US 2019/0365370 A1
(43) Pub. Date: Dec. 5, 2019

(54) LOCKING ALL-SUTURE ANCHOR ASSEMBLY AND METHOD FOR REPAIR

(71) Applicant: Raymond Thal, McLean, VA (US)

(72) Inventor: Raymond Thal, McLean, VA (US)

(21) Appl. No.: 16/425,418

(22) Filed: May 29, 2019

Related U.S. Application Data
(60) Provisional application No. 62/677,879, filed on May 30, 2018.
(60) Provisional application No. 62/677,907, filed on May 30, 2018.
(60) Provisional application No. 62/677,912, filed on May 30, 2018.

(51) Int. Cl. A61B 17/04; A61F 2/08

(52) U.S. Cl. CPC A61B 17/0401; A61F 2/0811; A61B 2017/0464; A61B 2017/044; A61B 2017/0414; A61B 2017/042; A61B 2017/0409; A61F 2002/0852; A61F 2002/0817

(57) ABSTRACT

An all-suture anchor assembly includes an all-suture anchor composed of at least one suture strand and an all-suture anchoring element threaded along the suture strand. The all-suture anchor assembly also includes a capture member integrated with the all-suture anchor for controlled capture of various elements, the capture member includes an elongated body having a forward first end, a rear second end, a catch member, and a lateral aperture with a suture passing therethrough.

### CROSS REFERENCE TO RELATED APPLICATION

This application claims the benefit of U.S. Provisional Patent Application Ser. No. 62/677,879, entitled “LOCKING ALL-SUTURE ANCHOR ASSEMBLY AND METHOD FOR REPAIR,” filed May 30, 2018, U.S. Provisional Patent Application Ser. No. 62/677,907, entitled “LOCKING ALL-SUTURE ANCHOR ASSEMBLY AND METHOD FOR REPAIR,” filed May 30, 2018, and U.S. Provisional Patent Application Ser. No. 62/677,912, entitled “LOCKING ALL-SUTURE ANCHOR ASSEMBLY AND METHOD FOR REPAIR,” filed May 30, 2018, all of which are incorporated herein by reference.

### BACKGROUND OF THE INVENTION

### 1. Field of the Invention

The present invention relates to devices and/or methods used in tissue repair, and more particularly to an all-suture anchor assembly and a method for attachment of biological tissue (i.e., tendons or ligaments) to a bone mass.

### 2. Description of the Related Art

Soft tissues, such as tendons and ligaments, generally are attached to bone by small collagenous fibers. These connections are strong but permit the tendons and ligaments to be flexible. When a tissue, or a portion of a tissue, is torn away from the bone and requires repair, a surgeon is often required to repair the detached soft tissue with sutures, which are passed through bone tunnels and tied. A number of devices have been developed for securing a ligament or tendon to a bone mass. These devices can be used in place of bone tunneling techniques. These attachment devices are usually applied through extensive surgical incisions and, in some circumstances, by arthroscopic surgical techniques. The placement of bone tunnels for repair can be difficult and generally requires large open incisions. Through the advent of arthroscopic surgery, where the surgeon looks into a joint cavity with an arthroscope, there has been a trend to repair soft tissues back to bone through small incisions called portals.

A variety of devices are available for attaching objects to bone, such as screws, staples, suture anchors, and sutures alone. These devices have been used to attach soft tissue, such as ligaments, tendons and muscles to bone. A suture anchor assembly is a device, which utilizes small anchors, including those made of suture material alone, with additional suture materials attached thereto. A device, such as a screw, is inserted into the bone mass and anchored in place. After insertion of the anchor device, the attached suture is passed through the tissue to be repaired. The tying of a knot in the suture is then required to secure the tissue to the bone. The process of passing the anchored suture through the soft tissue and tying a knot is time consuming and difficult to undertake in the tight space encountered during arthroscopic surgery and sometimes even during conventional open surgery.

Knotless anchor assemblies have been popular and are embodied in a number of prior patents, such as U.S. Pat. No. 5,709,708 wherein there is provided an assembly with an anchor means having a snag means and a loop suture element attached thereto. The suture loop is passed through the tissue to be repaired. The snag means then captures the loop suture element. The anchor is then inserted into a drill hole in a bone mass and the anchor locks into the bone. As the anchor is inserted into the drill hole, the tissue is pulled into secure attachment with a bone mass.

Further, in U.S. Pat. No. 6,045,574 there is provided an assembly with an anchor means having a snag means, and a hollow sleeve element with a loop suture element attached thereto. The snag means captures a loop suture element of the hollow sleeve element to draw tissue into secure attachment with a bone mass.

Further, there is provided an all-suture anchor assembly, such as disclosed in U.S. Patent Application Publication No. 2012/0290004, which is incorporated by reference, having an all fibrous construct. The device requires the tying of a knot to complete the surgical repair.

However, difficulties still exist with the all-suture anchor assembly and the present invention attempts to address these with a method and apparatus for adjustable, knotless anchoring using an all-suture anchor assembly.

### SUMMARY OF THE INVENTION

It is, therefore, an object of the present invention to provide an all-suture anchor assembly including an all-suture anchor composed of at least one suture strand and an all-suture anchoring element threaded along the suture strand. A capture member is integrated with the all-suture anchor for controlled capture of various elements. The capture member includes an elongated body having a forward first end, a rear second end, a catch member, and a lateral aperture with a suture passing therethrough.

It is also an object of the present invention to provide an all-suture anchor assembly wherein the at least one suture strand has a suture first end and a suture second end which are secured together at an intersection.

It is another object of the present invention to provide an all-suture anchor assembly wherein the catch member is a lateral slot positioned at the forward first end of the capture member.

It is a further object of the present invention to provide an all-suture anchor assembly wherein the rear second end of the capture member is shaped and dimensioned for selective coupling with a delivery inserter.

It is also an object of the present invention to provide an all-suture anchor assembly including a delivery inserter having an elongated body with a proximal first end provided with a handle for actuation and a distal second end shaped and dimensioned for selective attachment to capture member.

It is another object of the present invention to provide an all-suture anchor assembly wherein the elongated body includes a slot extending from the distal second end to a central position along the length of the elongated body, wherein the slot is shaped such that the all-suture anchor may be passed therethrough.

It is a further object of the present invention to provide an all-suture anchor assembly wherein a portion of the elongated body proximal to the slot is provided with an elongated cannulation extending from the slot to the proximal first end, and the cannulation is shaped and dimensioned to receive an actuation rod that engages with the all-suture anchor.

It is also an object of the present invention to provide an all-suture anchor assembly wherein the at least one suture strand is an adjustable suture strand.

It is another object of the present invention to provide an all-suture anchor assembly wherein the at least one suture strand is threaded through the all-suture anchoring element.

It is a further object of the present invention to provide an all-suture anchor assembly including a suture loop.

It is also an object of the present invention to provide an all-suture anchor assembly including an all-suture anchor composed of at least one suture strand and an all-suture anchoring element threaded along the suture strand. The all-suture anchor assembly also includes a capture member or islet tip, and a delivery inserter including an elongated body with a proximal first end and a distal second end shaped and dimensioned for selective attachment to the capture member or the islet tip. The elongated body includes a slot extending from the distal second end to a central position along the length of the elongated body, wherein the slot is shaped such that the all-suture anchor may be passed therethrough. A portion of the elongated body proximal to the slot is provided with an elongated cannulation extending from the slot to the proximal first end, wherein the cannulation is shaped and dimensioned to receive an actuation rod that engages with the all-suture anchor.

It is another object of the present invention to provide an all-suture anchor assembly wherein the capture member or islet tip is a capture member including a catch member.

It is a further object of the present invention to provide an all-suture anchor assembly wherein the capture member or islet tip is an islet tip islet tip having a body with an aperture formed therein.

It is also an object of the present invention to provide an all-suture anchor assembly including a plurality of elongated sutures passing through the aperture formed in the islet tip.

It is another object of the present invention to provide an all-suture anchor assembly including a bone anchor secured to the plurality of elongated sutures.

It is a further object of the present invention to provide an all-suture anchor assembly wherein the slot defines first and second legs.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 shows a top plan view of an all-suture anchor and passing-suture in accordance with an embodiment of the present invention.

FIGS. 1A and 1B show alternate embodiments of an all-suture anchor and passing-suture in accordance with the present invention.

FIGS. 2, 3 and 4 show operation and usage of an all-suture anchor assembly in accordance with the embodiment shown with reference to FIG. 1.

FIG. 5 is a top plan view of an alternate embodiment of an all-suture anchor and passing-suture.

FIGS. 6-8 disclose the various steps of the usage and operation of the all-suture anchor and passing-suture shown in FIG. 5.

FIG. 9 shows use of an all-suture anchor assembly in accordance with an alternate embodiment.

FIG. 10 shows an all-suture anchor assembly in accordance with another embodiment of the present invention.

FIGS. 11, 12 and 13 show the usage and operation of the all-suture assembly shown with reference to FIG. 10.

FIG. 14 shows operation of an all-suture anchor assembly in accordance with an alternate embodiment.

FIG. 15 shows the usage and operation of an alternate embodiment of an all-suture anchor assembly.

FIG. 16 shows a further embodiment of an all-suture anchor assembly.

FIG. 17 is a view of an all-suture anchor assembly in accordance with an embodiment of the present invention.

FIGS. 18, 19 and 20 show usage and operation of the all-suture anchor assembly shown in FIG. 17.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

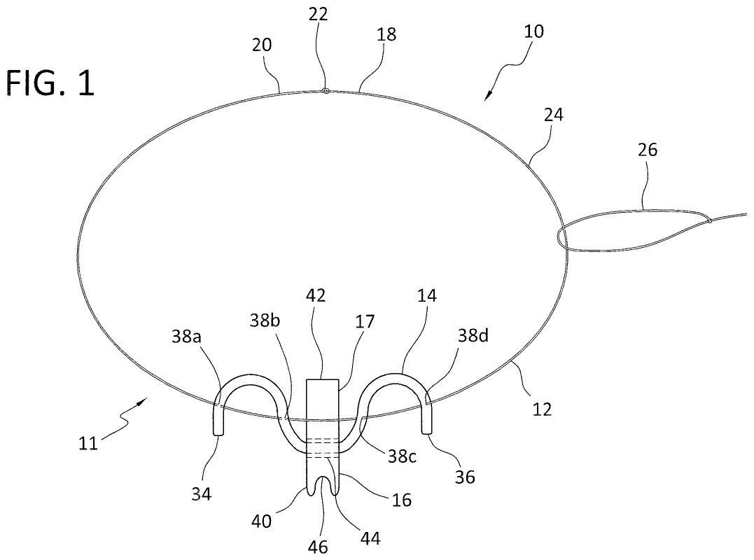

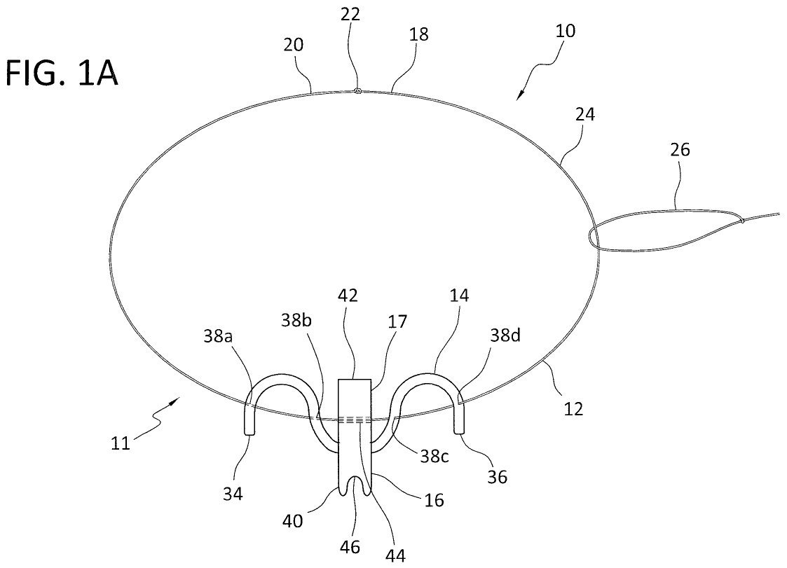

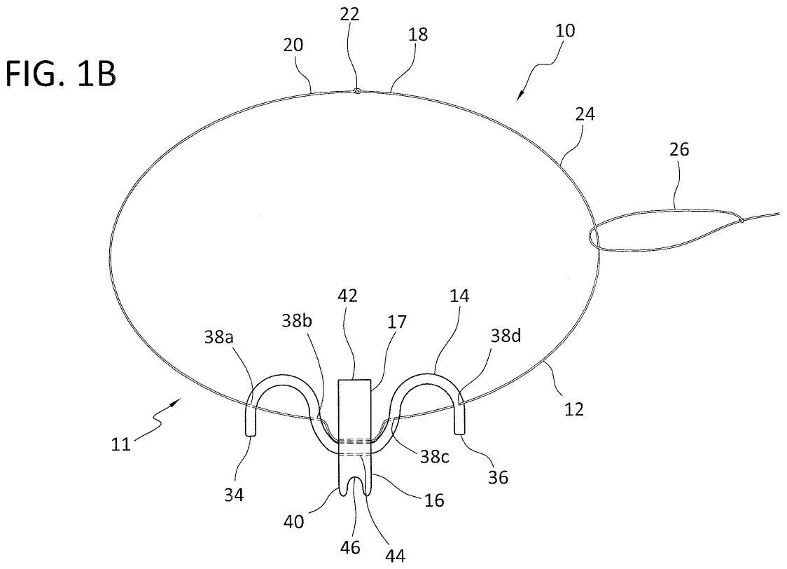

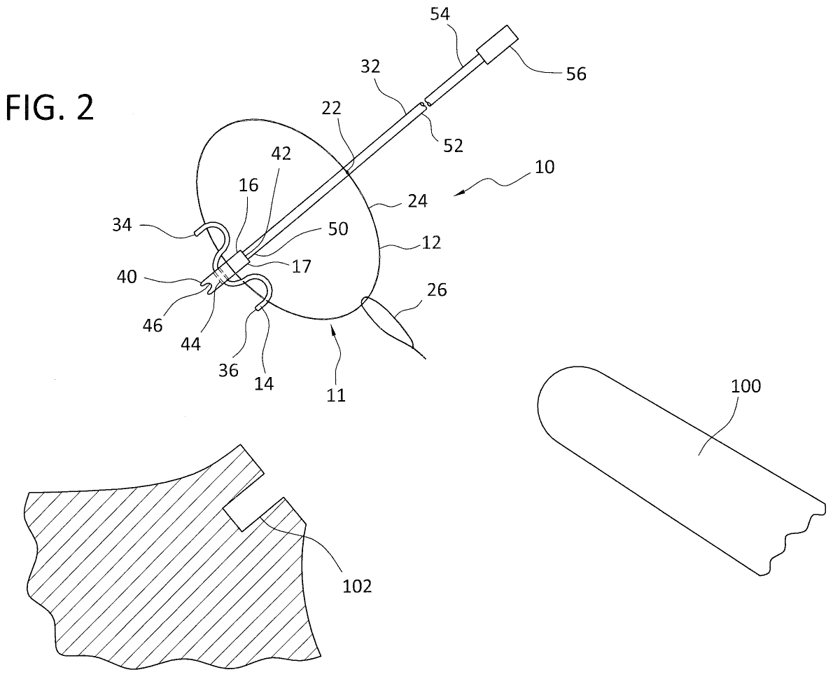

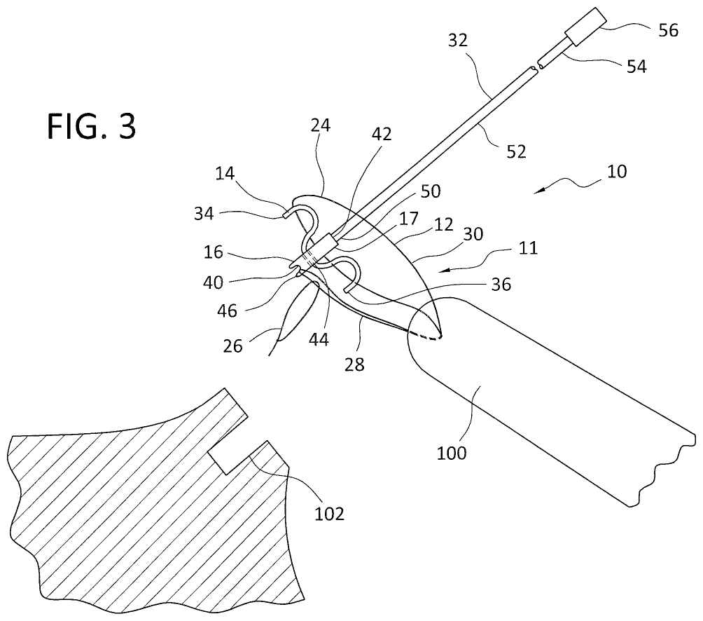

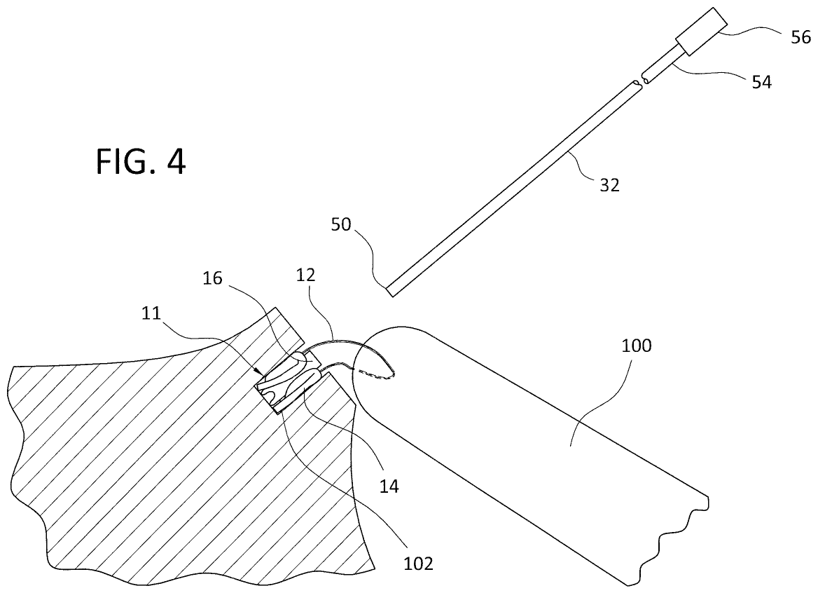

In accordance with the present invention, and with reference to FIGS. 1 to 4, an all-suture anchor assembly 10 and associated method are disclosed. The all-suture anchor assembly 10 includes an all-suture anchor 11 composed of at least one suture strand 12 and an all-suture anchoring element 14 threaded along the suture strand 12 in manner discussed below in greater detail. The all-suture anchor assembly 10 also includes a capture member 16 integrated with the at least one suture strand 12 or the all-suture anchoring element 14 for controlled capture of various elements in a manner discussed below in greater detail. The suture strand 12 has a suture first end 18 and a suture second end 20 which are secured together at an intersection 22 (knot, bead, enlargement, weaved suture engaging element (i.e.—Chinese fingertrap element), or other capturable structure).

As will be discussed below in greater detail, with the suture first end 18 and the suture second end 20 coupled at the intersection 22 to define a suture loop 24, the portion of the suture loop 24 opposite the all-suture anchoring element 14 is pulled through the tissue 100 using a passing suture 26. This creates a partial suture loop 28 on one side of the tissue 100 and a partial all-suture anchoring element loop 30 on the other side of the tissue 100. The capture member 16, to which the delivery inserter 32 is attached, is then used to capture the partial suture loop 28 prior to insertion into a bone hole 102, as will be discussed below in detail.

The all-suture anchoring element 14 is preferably composed solely of an enlarged piece of cylindrical suture material or a suture tape. The enlarged surface area of the all-suture anchoring element 14 allows for the passage of the suture strand 12 therethrough in a manner providing for entanglement of the all-suture anchor element 14 and the suture strand 12. The suture anchor element 14 includes a first end 34 and a second end 36, as well as a thickness, a width and a length along a longitudinal axis. As briefly mentioned above, the all-suture anchor assembly 10 includes the suture strand 12, which is passed through, or weaved through, the all-suture anchoring element 14 at various locations along the length of the all-suture anchoring element 14 (so as to define the all-suture anchor 11). That is, the suture strand 12 penetrates and traverses the all-suture anchoring element 14 so as to define apertures in the all-suture anchoring element 14. The intersections of the suture strand 12 with the all-suture anchoring element 14 are referred to herein as suture aperture locations 38a-d and, as such, each of the suture aperture locations 38a-d is a location where the suture strand 12 passes through the thickness of the all-suture anchoring element 14. As shown in FIG. 1, there are four suture aperture locations 38a-d. It is appreciated that as few as two suture aperture locations on a particular all-suture anchor assembly may function well. Similarly, more suture aperture locations may be provided. While the suture aperture locations 38a-d disclosed above in accordance with a preferred embodiment are all centrally located along the all-suture anchoring element 14 so as to be oriented along the central longitudinal axis of the all-suture anchoring element 14, it is appreciated the suture aperture locations 38a-d may be varied (for example, staggered on opposite sides of the central longitudinal axis of the all-suture anchoring element 14) without departing from the spirit of the present invention. In accordance with a preferred embodiment, the all-suture anchoring element 14 may have various length and width dimensions depending upon the purpose for which it is intended.

While the suture strand is disclosed above as being threaded through the all-suture anchoring element, it is appreciated that suture strand could be passed through a central passageway defined by the all-suture anchoring element where the all-suture anchoring element is constructed as an elongated cylindrical member. Similar, and while the embodiment above discloses the all-suture anchoring element as having separate ends, the ends of the all-suture anchoring element could certainly be connected as is known in the art.

With the foregoing in mind, it is appreciated that a large variety of constructions and materials will work for the all-suture anchoring element 14. For example, the all-suture anchor may take a variety of forms as known in the art, for example, ConMed Y-Knot® & Y-Knot® RC, Parcus Draw Tight™, Smith & Nephew Suturefix Ultra, Smith & Nephew Q-FIX®, Zimmer Biomet: JuggerKnot® Soft Anchor; Cayenne Medical (Zimmer Biomet company) SureLock®, and Stryker® Iconix. It has been discovered that for each type of construction (i.e., braided, woven, non-woven, or knitted) there is an advantage for using a material that increases in width for every reduction in length. This advantage provided for increased diameters for a particular number of folds, pleats, crinkles or other changes in the shape of the all-suture anchor element. Regardless of the material chosen for use in accordance with the present invention, the material must exhibit desirable deformation and retention characteristics.

The all-suture anchor assembly 10 further includes the solid capture member 16 that is integrated onto the all-suture anchoring element 14. Those skilled in the art will appreciate, the capture member may be composed of a variety of biocompatible materials (for example, biocompatible polymers) known to those skilled in the art. In accordance with a preferred embodiment, the solid capture member 16 includes an elongated body 17 having a forward first end 40 and a rear second end 42. The capture member 16 is preferably secured to the all-suture anchoring element 14 by passing the all-suture anchoring element 14 through a lateral aperture 44 formed in the capture member 16. While FIGS. 1-4 disclose the all-suture anchoring element 14 passing through the capture member 16, it is appreciated other mechanisms for integrating the capture member 16 with the suture strand 12 and the all-suture anchoring element 14 may be employed. For example, and with reference to FIGS. 1A and 1B, the suture strand 12 might be passed through the lateral aperture 44 (FIG. 1A) or both the suture strand 12 and the all-suture anchoring element 14 may be passed through the lateral aperture (FIG. 1B). Functionality in conjunction with the capture member 16 may be further enhanced by the provision of spikes or ridges along the outer surface thereof.

The forward first end 40 also includes a catch member 46. In accordance with a disclosed embodiment, the catch member 46 is a lateral slot positioned at the forward first end 40 of the capture member 16. While the lateral slot is shown in accordance with the disclosed embodiment, it is appreciated the lateral slot could be positioned at various locations along the capture member so long as the lateral slot is able to engage the suture strand 12 for operation in accordance with the present invention. The catch member 46 is shaped and dimensioned for capture and retention of suture(s), and other surgical elements, as discussed below in greater detail. While the catch member 46 is disclosed herein as being a lateral slot, it is appreciated the catch member 46 may take a variety of forms so long as it is capable of catching or snagging the suture strand 12, and therefore may take various shapes and have various dimensions without departing from the spirit of the present invention; for example, the catch member 46 may be shaped in the form of a hook, or other type projection, or a recess cut into the capture member, or a slit cut into an existing opening in the capture, for engaging the continuous loop of a suture strand.

The rear second end 42 of the capture member 16 is shaped and dimensioned for selective coupling with a distal second end 50 of the delivery inserter 32, for example, via a threaded attachment structure. In accordance with a preferred embodiment, the delivery inserter 32 includes an elongated body 52 with a proximal first end 54 and a distal second end 50. The proximal first end 54 is provided with a handle 56 for actuation in accordance with the present invention. The distal second end 50 is shaped and dimensioned for selective attachment to capture member 16. Such delivery inserters, and in particular, compression mechanisms are known within the art, for example, see the Draw Tight™ Suture Based Anchor System as manufactured by Parcus, and various compression mechanisms may be used within the spirit of the present invention.

The delivery inserter 32 is operated by a surgeon whereby the surgeon manipulates the all-suture anchoring element 14 and the suture strand 12. The surgeon inserts the all-suture anchoring element 14 and the entangled portion of the suture strand 12 in the bone hole 102 by pushing the distal second end 50 of the delivery inserter 32 into the bone hole 102. At that point, the surgeon can push a button or turn a device on the delivery inserter 32 which enables the deployment of the all-suture anchor element 14. While a push-button or twisting mechanism is disclosed above for deployment of the all-suture anchor element 14, it is appreciated other known mechanisms (for example, pulling a deployment suture) for all-suture anchors may be employed without departing from the spirit of the present invention. The all-suture anchoring element 14 is then folded, bent, creased, crinkled, bunched or otherwise changed in shape after it is inserted into the bone hole 102 in a manner that increases the size of the all-suture anchoring element 14 in a direction substantially perpendicular to the longitudinal axis of the bone hole 102 to develop an outwardly directed forced that is directed at the walls of the bone hole 102. The folding, bending, creasing, crinkling, bunching or other changes in the shape of the all-suture anchoring element 14 is achieved using known techniques. As shown, and further to the discussion above, with the suture first end 18 and the suture second end 20 coupled at the intersection 22 to define a suture loop 24, the portion of the suture loop 24 opposite the all-suture anchoring element 14 is pulled through the tissue 100 using a passing suture 26. This creates the partial suture loop 28 on one side of the tissue 100 and the partial all-suture anchoring element loop 30 on the other side of the tissue 100. The capture member 16, to which the delivery inserter 32 is attached, is then used to capture the partial suture loop 28 prior to insertion into a bone hole 102.

The suture strand 12, the all-suture anchoring element 14, and the capture member 16 are installed within the bone hole 102 in an elongated fashion to take advantage of a small diameter configuration, referred to herein as an undeployed state or installation state. Deployment occurs after the delivery inserter 32 is manipulated such that the capture member 16 captures and retains suture strands, and other surgical elements. This capture and retention are followed by positioning of the suture strand 12, the all-suture anchoring element 14, and the capture member 16 within a bone hole 102 for retention of the various elements to a bone mass.

Thereafter, the all-suture anchoring element 14 is deployed causing the all-suture anchoring element 14 to fold, bend, crease, crinkle, bunch or otherwise change shape in a manner that compresses the all-suture anchoring element 14. It is appreciated that deployment may occur before, after, or as the delivery inserter 32 is removed. As the all-suture anchoring element 14 is compressed in this manner it ultimately increases in size in a direction substantially perpendicular to the longitudinal axis of the bone hole 102 (or otherwise oriented to contact side walls of the bone hole 102) and develops an outwardly directed forced that is directed at the walls of the bone hole 102. As such, and after the delivery inserter 32, or other delivery instrument, has been removed, the all-suture anchoring element 14 exhibits an outward bias resulting in expansion of the all-suture anchoring element 14 in a direction substantially perpendicular to the longitudinal axis of the bone hole 102 (or otherwise oriented to contact side walls of the bone hole 102) into which it is positioned, resulting in frictional engagement or gripping the wall of the bone, which is referred to herein as the expanded deployed state.

The all-suture anchoring element 14 is folded or otherwise compressed to form pleats between adjacent suture aperture locations 38a-d. This pleating reduces the distance between the first suture aperture location and the second suture aperture location, as measured along the length of the all-suture anchoring element 14. These pleats form a bunched mass of suture material effectively increasing a diameter, or cross sectional dimension, (as measured in relation to the axis of the bone hole 102) of the all-suture anchoring element 14, which ultimately causes the all-suture anchor assembly 10 to displace cancellous bone. The relative increase in the cross sectional size of the all-suture anchoring element 14 in the direction substantially perpendicular to the longitudinal axis of the bone hole 102 (or in another direction to facilitate contact of the all-suture anchoring element 14 with the side walls of the bone hole 102) creates a retention force of the all-suture anchor assembly 10.

It is appreciated that a mechanical tensioning mechanism, as is well known to those skilled in the art, may be used during the deployment of the all-suture anchoring element. Such mechanical tensioning mechanisms pull or ratchet the suture while the delivery inserter holds the all-suture anchoring element in place. Mechanical tensioning, of this nature, may be preferable as this can more tightly ‘fold’ or ‘bunch’ the all-suture anchoring element, thereby increasing the created tension, that is, the outward force of the all-suture anchoring element, which is relative to the longitudinal axis of the bone hole and toward the walls of the bone hole, resulting from increased compression of the all-suture anchoring element. The changed shape of the all-suture anchoring element provides security within the bone, for example, below the cortical layer.

It should be appreciated that the relative fit of the all-suture anchor assembly 10 in the bone hole 102 in its deployed configuration is shown as being relatively “loose.” This is done to provide a clear view for the elements making up the present invention. In practice, it is appreciated that the suture, all-suture anchoring element 14, the capture member 16, a portion of the suture strand 12, and delivery inserter 32 would be tightly pressed into the bone hole 102, as any excess space would need to be taken up by the expansion of the all-suture anchor assembly 10 in a direction substantially perpendicular to the longitudinal axis of the bone hole 102 (or otherwise oriented to contact side walls of the bone hole 102).

In accordance with an alternate embodiment, and as described in U.S. Patent Application Publication No. 2017/0290578, entitled “Modified Adjustable, Locking All-Suture Anchor Assembly and Method For Repair,” which is incorporated herein by reference, a sleeve may be used to enhance anchoring of the all-suture anchoring element 14 within the bone hole 102. This is particularly valuable when soft bone or bone cysts are encountered that might reduce the security achieved by the deployed all-suture anchor.

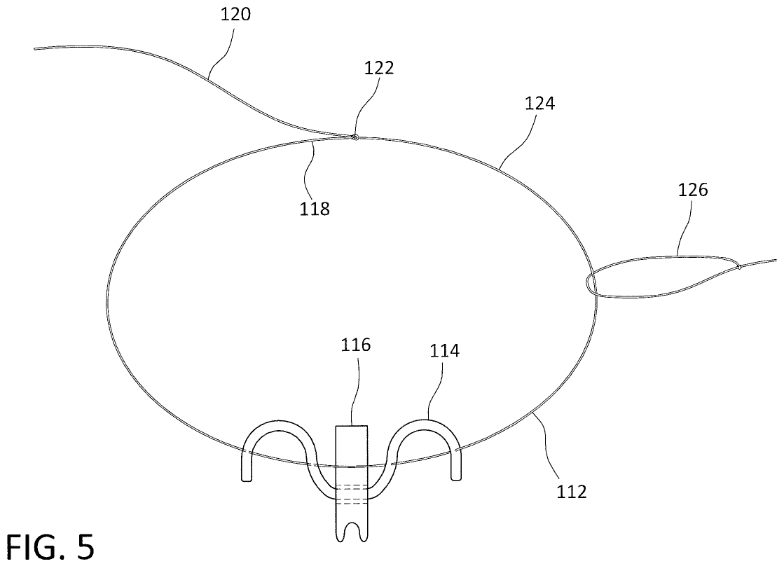

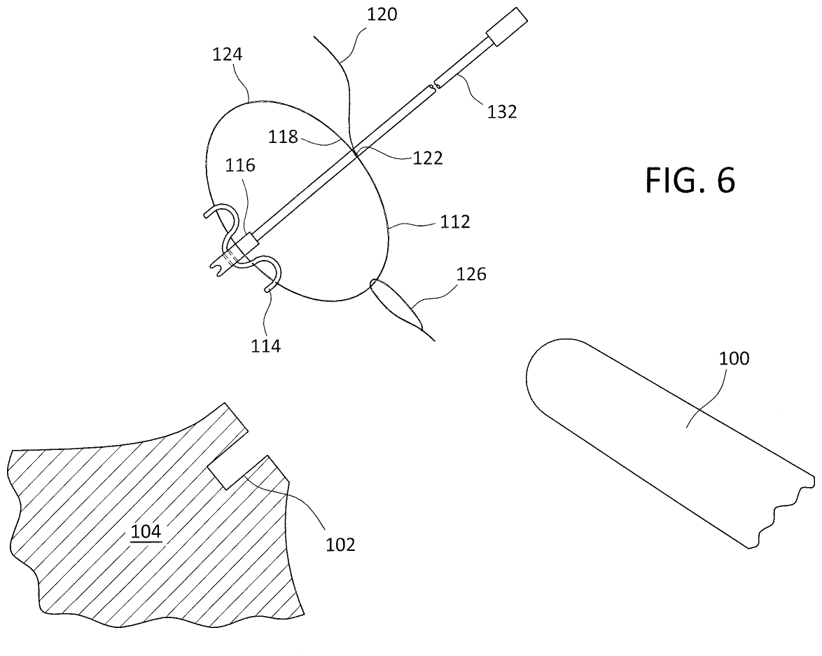

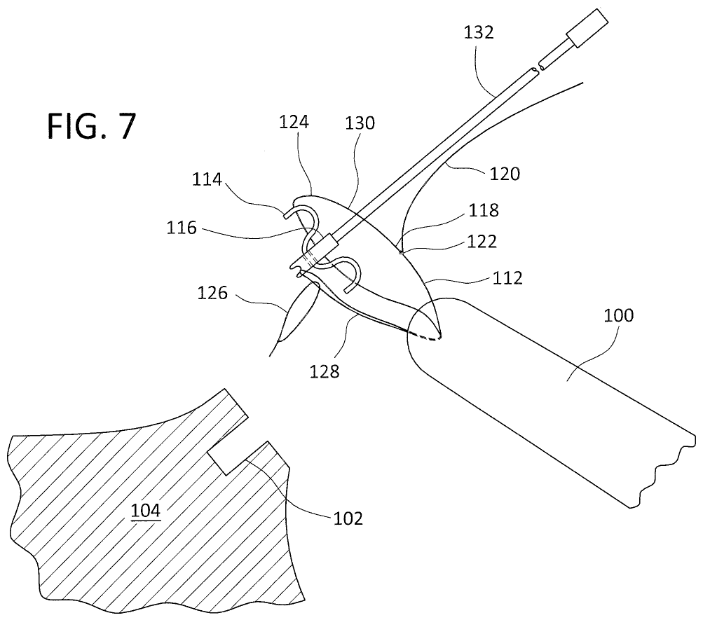

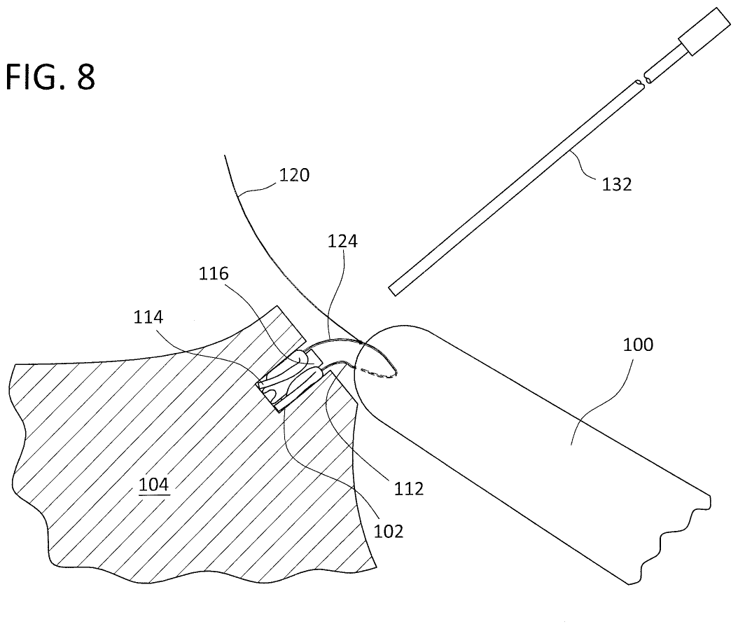

The previous description described a suture strand 12 defining a loop of a fixed length. However, the suture strand may be formed as an adjustable suture loop having a length capable of controlled adjustment; that is, the suture loop formed by the suture strand may be decreased in size in a controlled manner. With reference to FIGS. 5 to 8, an all-suture anchor assembly 110 includes an adjustable suture strand 112 formed with an adjustment suture second end 120 (which is an extension of the suture strand 112) such that upon pulling of the adjustment suture second end 120 of the adjustable suture strand 112 the adjustable suture loop 124 defined by the adjustable suture strand 112 is shrunk allowing for tissue to be pulled toward the all-suture anchoring element 114. In particular, the adjustable suture strand 112 has a suture first end 118 and an adjustment suture second end 120. The suture first end 118 includes a slip knot 122 (or other coupling mechanism as described above and known to those skilled in the art), while the adjustment suture second end 120 is passed through the slip knot 122 to create the adjustable suture loop 124. The adjustment suture second end 120 therefore extends beyond the slip knot 122 for access as described below.

The suture first end 118 is held in or by the all-suture anchoring element 114 through the inclusion of the slip knot 122. With the suture first end 118 held in position relative to the all-suture anchoring element 114, the adjustment suture second end 120 is threaded through the all-suture anchoring element 114 and is then passed through the slip knot 122.

With the exception of the inclusion of the slip knot 122 for allowing reduction in the size of the adjustable suture loop 124, operation proceeds substantially the same as described above. The portion of the adjustable suture loop 124 opposite the all-suture anchoring element 114 is pulled through the tissue 100 using a passing suture 126. This creates a partial suture loop 128 on one side of the tissue 100 and a partial all-suture anchoring element loop 130 on the other side of the tissue 100. The capture member 116, to which the delivery inserter 132 is attached, is then used to capture the partial suture loop 128 for insertion into a bone hole 102. While a passing suture 126 is disclosed for use in accordance with a preferred embodiment, the passing suture 126 could be replaced by the adjustment suture second end 120; that is, the adjustment suture second end 120 would be passed through the tissue 100 in order to properly position the suture strand 112, the all-suture anchoring element 114, and the capture member 116 as described above.

The suture strand 112, the all-suture anchoring element 114, and the capture member 116 are installed within the bone hole 102 in an elongated fashion to take advantage of a small diameter configuration, referred to herein as an undeployed state or installation state. Deployment occurs after the delivery inserter 132 is manipulated such that the capture member 116 captures and retains suture strands, and other surgical elements. This capture and retention are followed by positioning of the suture strand 112, the all-suture anchoring element 114, and the capture member 116 within a bone hole 102 for retention of the various elements to a bone mass 104.

Thereafter, the all-suture anchoring element 114 is deployed by the delivery inserter 132 causing the all-suture anchoring element 114 to fold, bend, crease, crinkle, bunch or otherwise change shape in a manner that compresses the all-suture anchoring element 114. It is appreciated that deployment may occur before, after, or as the delivery inserter 132 is removed. As the all-suture anchoring element 114 is compressed in this manner it ultimately increases in size in a direction substantially perpendicular to the longitudinal axis of the bone hole 102 (or otherwise oriented to contact side walls of the bone hole 102) and develops an outwardly directed forced that is directed at the walls of the bone hole 102. As such, and after the delivery inserter 132, or other delivery instrument, has been removed, the all-suture anchoring element 114 exhibits an outward bias resulting from the expansion of the all-suture anchoring element 114 in a direction substantially perpendicular to the longitudinal axis of the bone hole 102 (or otherwise oriented to contact side walls of the bone hole 102) into which it is positioned, resulting in frictional engagement or gripping the wall of the cancellous bone, which is referred to herein as the expanded deployed state.

With the all-suture anchoring element 114 securely held within the bone hole 102, the adjustment suture second end 120 may be pulled to further draw the tissue 100 to the bone 104 due to the reduction in the size of the suture loop 124. This described adjustment in the size of the adjustable suture loop 124 can be performed before, during or after suture strand 112 is captured by the catch-means of capture member 116. This adjustment can also be performed before, during, or after insertion into bone or anchor sleeve.

Although a single hole (single row) repair is described above, it is appreciated that more extensive repairs can comprise multiple drilled bone holes. When multiple drilled bone holes are used, multiple adjustable, locking all-suture anchor assemblies are used. Each assembly is used as described above and inserted into a separate drilled bone hole or a mounting sleeve placed in the drilled bone hole. This enables a surgeon to suture various sections of a tissue and draw the tissue to bone at several locations to secure the tissue for a repair.

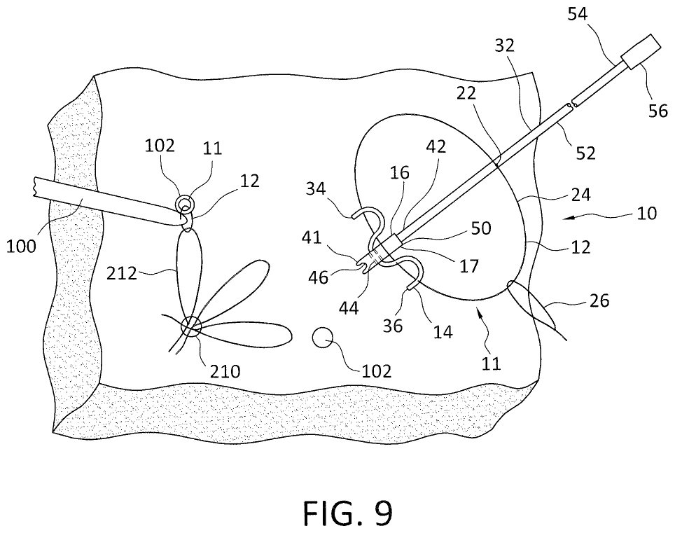

For example, and with reference to FIG. 9, the all-suture anchor assembly 10 (although the embodiment shown with reference to FIGS. 1 to 4 is shown in FIG. 9 it is appreciated the embodiment shown with reference to FIGS. 5 to 8 may also be used) described above is used in performing a multi-row repair wherein a medial anchor 210 with one or a plurality of multiple adjustable loop(s) 212 is used in conjunction with the present all-suture anchor assembly 10. The adjustable loops 212 are preferably suture loops constructed with a slip knot allowing the effective size of the loop to be adjusted in a manner known to those skilled in the art. In accordance with such an embodiment, the medial adjustable loop(s) 212 is captured by the all-suture anchor assembly 10 in the same way in which a section of tissue 100 is captured. The all-suture anchor 11, 111 is then inserted into separate lateral drill holes 102. The medial loop(s) 212 can then be adjusted to tension the repair. It is further appreciated, the loop of the all-suture anchor 11 can further be through tissue 100 and captured by another all-suture anchor in accordance with the present invention so as to create a linked chain repair.

With the inclusion of a solid capture member as discussed above, the methodology may be altered considering a bone hole may not be required, and the solid capture member may be used to penetrate the bone mass and form a cavity in the bone into which the all-suture anchor assembly is positioned. In accordance with such an embodiment, and with the delivery inserter directly attached to the solid capture member, the forward first end of the solid capture member is impacted directly into bone mass, with the remainder of the solid capture member following and being inserted into the bone mass. In such an embodiment, it might be preferable to position the catch member on the side of the solid capture member to reduce the risk of suture abrasion during anchor insertion. Thereafter, the all-suture anchoring element and the anchor suture strand are forced into the hole created by the solid capture member, and the all-suture anchoring element is folded, bent, creased, crinkled, bunched or otherwise changed in shape in a manner that compresses the all-suture anchoring element as discussed above. Other than the inclusion of the solid capture member and the direct impacting into the bone mass, the methods for use disclosed herein remain the same.

The all-suture knotless repair system described above may be employed in a variety of medical procedures. Included amongst those medical procedures that may take advantage of the present all-suture knotless repair system are those procedures disclosed in U.S. Patent Application Publication No. 2015/0216522, entitled “SUTURE ANCHOR,” published Aug. 6, 2015, which is incorporated herein by reference. Briefly, and considering the procedures disclosed in the '522 publication, it is important to note that 1) one or more medial row anchors can be used; 2) one or more lateral row anchors can be used; and 3) all, or only some, of the sutures from one medial row anchor can be linked to one, or more than one, lateral row anchor (therefore, sutures form multiple medial row anchors that may be crossed to multiple lateral row anchors).

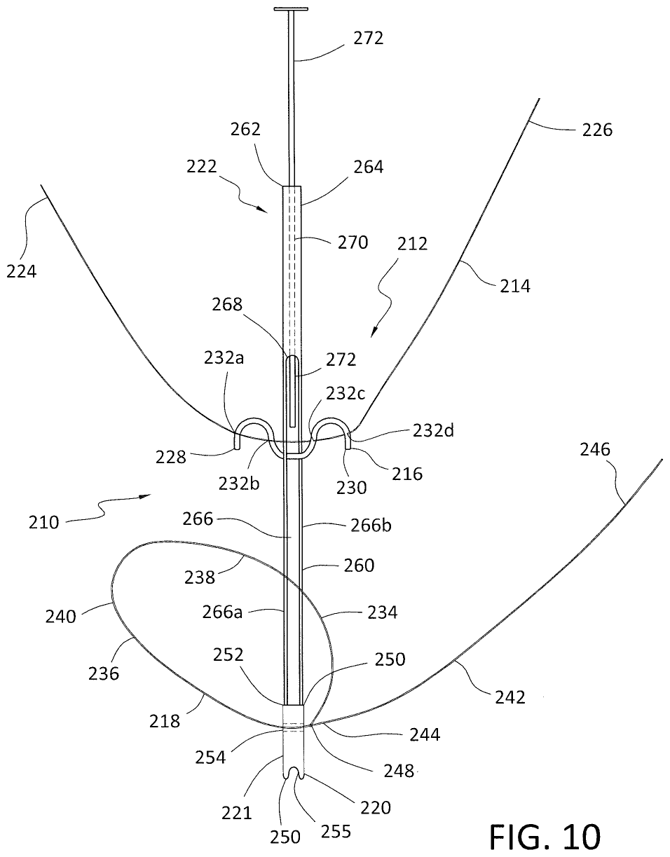

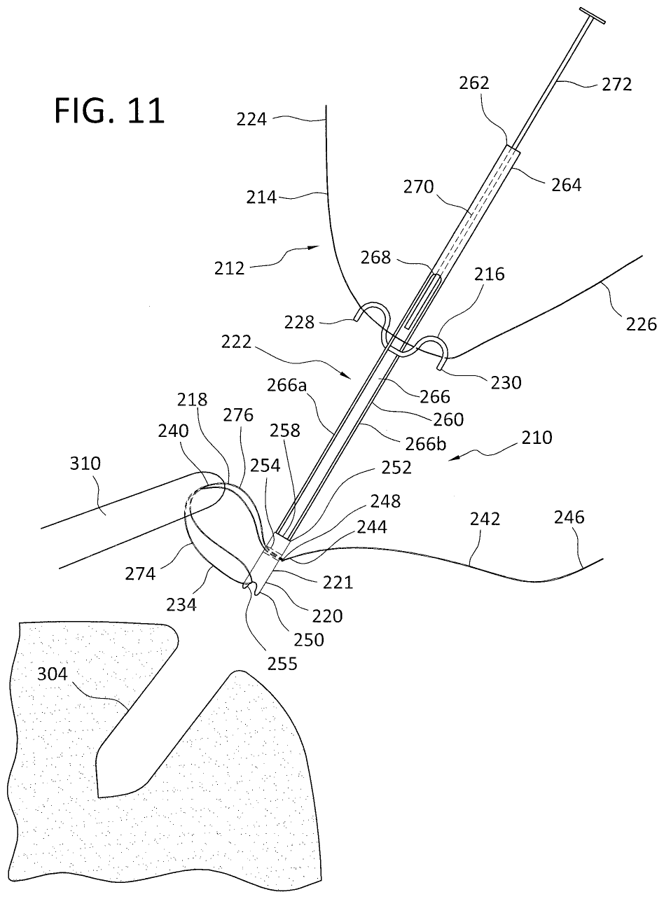

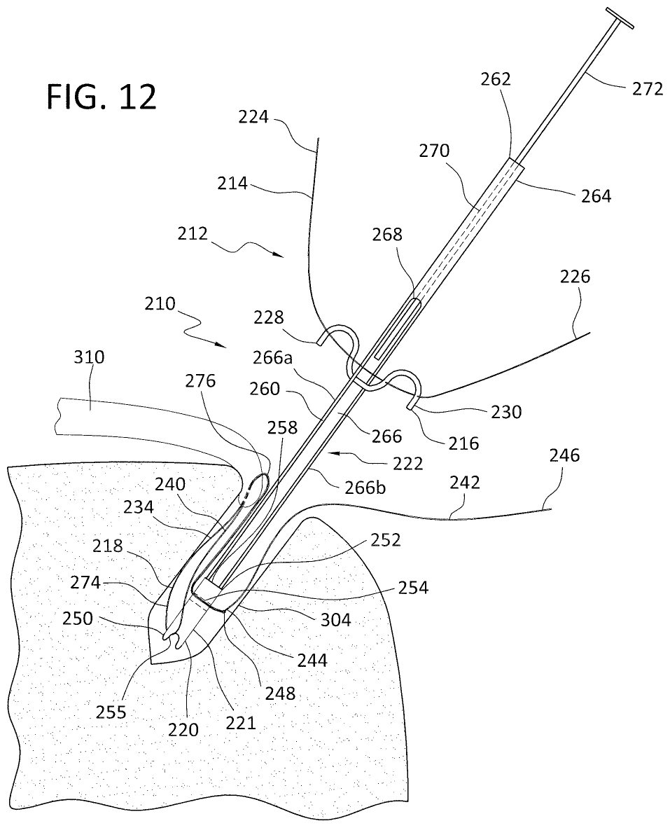

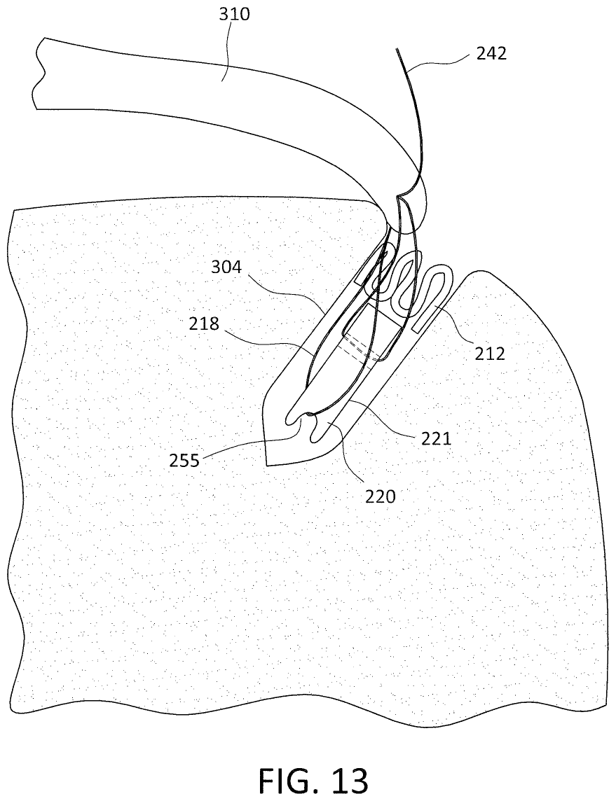

In accordance with another embodiment, and with reference to FIGS. 10 to 13, an all-suture anchor assembly 210 and associated method are disclosed. The all-suture anchor assembly 210 includes an all-suture anchor 212 composed of a suture strand 214 and an all-suture anchoring element 216 threaded along the suture strand 214 in a manner discussed below in greater detail. The all-suture anchor assembly 210 also includes an adjustable suture loop 218, a capture member 220 integrated with the adjustable suture loop 218, and a delivery inserter 222.

As with the prior embodiment disclosed above with reference to FIGS. 1 to 9, it should be appreciated that while a preferred all-suture anchor 212 is disclosed below in accordance with a preferred embodiment, the all-suture anchor 212 may take a variety of forms as known in the art. The suture strand 214 has a suture first end 224 and a suture second end 226. The all-suture anchoring element 216 is preferably composed solely of an enlarged piece of cylindrical suture material or a suture tape. The enlarged surface area of the all-suture anchoring element 216 allows for the passage of the suture strand 214 therethrough in a manner providing for entanglement of the all-suture anchor element 216 and the suture strand 214. The suture anchor element 216 includes a first end 228 and a second end 230, as well as a thickness, a width and a length along a longitudinal axis.

As briefly mentioned above, the all-suture anchor assembly 210 includes the suture strand 214, which is passed through the all-suture anchoring element 216 at various locations along the length of the all-suture anchoring element 216. That is, the suture strand 214 penetrates and traverses the all-suture anchoring element 216 so as to define apertures in the all-suture anchoring element 216. The intersections of the suture strand 214 with the all-suture anchoring element 216 are referred to herein as suture aperture locations 232a-d and, as such, each of the suture aperture locations 232a-d is a location where the suture strand 214 passes through the thickness of the all-suture anchoring element 216. As shown in FIG. 10, there are four suture aperture locations 232a-d. It is appreciated that as few as two suture aperture locations on a particular all-suture anchor assembly may function well. Similarly, more suture aperture locations may be provided. While the suture aperture locations 232a-d disclosed above in accordance with a preferred embodiment are all centrally located along the all-suture anchoring element 216 so as to be oriented along the central longitudinal axis of the all-suture anchoring element 216, it is appreciated the suture aperture locations 232a-d may be varied (for example, staggered on opposite sides of the central longitudinal axis of the all-suture anchoring element 216) without departing from the spirit of the present invention. In accordance with a preferred embodiment, the all-suture anchoring element 216 may have various length and width dimensions depending upon the purpose for which it is intended.

While the suture strand 214 is disclosed above as being threaded through the all-suture anchoring element 216, it is appreciated that suture strand 214 could be passed through a central passageway defined by the all-suture anchoring element 216 where the all-suture anchoring element 216 is constructed as an elongated cylindrical member. Similar, and while the embodiment above discloses the all-suture anchoring element 216 as having separate ends, the ends of the all-suture anchoring element 216 could certainly be connected as is known in the art.

With the foregoing in mind, and as with the embodiment disclosed above with reference to FIGS. 1 to 9, it is appreciated that a large variety of constructions and materials will work for the all-suture anchor 212. It has been discovered that for each type of construction (i.e., braided, woven, non-woven, or knitted) there is an advantage for using a material that increases in width for every reduction in length. This advantage provides for increased diameters for a particular number of folds, pleats, crinkles or other changes in the shape of the all-suture anchoring element 16. Regardless of the material chosen for use in accordance with the present invention, the material must exhibit desirable deformation and retention characteristics.

The all-suture anchor assembly 210 also includes an adjustable suture loop 218. The adjustable suture loop 218 is composed of one or more strands of suture 234. The adjustable suture loop 218 includes a first segment 236 and a second segment 238 linked so as to form a loop member 240 and one or more tensioning suture components 242 extending from the loop member 240. It is appreciated that the tensioning suture component(s) 242 is preferably a continuation of one of the first segment 236 or the second segment 238 of adjustable loop 218. In accordance with a preferred embodiment, the first segment 236 or the second segment 238 may be linked by a slip knot 248, but they may be linked in several ways. For example, they can be linked using a sliding/locking knot or one of the sutures can be passed through a weaved attachment on the other suture (like a Chinese finger trap) so that the suture loop 218 can be reduced in size by pulling on one of the tensioning suture component(s) 242. As such, the tensioning suture component 242 may be composed of more than one suture depending upon the number of strands of suture used in the construction of the adjustable suture loop 218. The tensioning suture component 242 includes a first end 244 and a second end 246. The first end 244 is secured to the loop member 240 at the slip knot 248 defining the loop member 240, while the second end 246 freely extends therefrom for manipulation by the medical practitioner as discussed below in greater detail. While a slip knot is disclosed herein in accordance with the disclosed embodiment, it is appreciated as described above that other knot designs and adjustable coupling structures, for example, a weaved portion which functions similar to a Chinese finger trap, might be used. Further, it is appreciated that the adjustable suture loop discussed above may be replaced with, or used in combination with, a fixed length suture loop.

The adjustable suture loop 218 is constructed by tying the first segment 236 and the second segment 238 of the strand of suture 240 with a sliding or slip knot 248. By constructing the adjustable flexible suture loop 218 with a slip knot 248, one can alter the size of the loop member 240 by pulling upon the tensioning suture component 242, which functions by pulling a portion of the suture strand 240 through the slip knot 248 and ultimately reducing the size of the loop member 240 while increasing the length of the tensioning suture component 242.

The all-suture anchor assembly 210 further includes the solid capture member 220 that is integrated onto the adjustable suture loop 218. Those skilled in the art will appreciate the capture member may be composed of a variety of biocompatible materials, for example, biocompatible polymers, known to those skilled in the art. In accordance with a preferred embodiment, the solid capture member 220 includes an elongated body 221 having a forward first end 250 and a rear second end 252. In accordance with a preferred embodiment, the capture member 220 has flattened sides to allow the suture to pass on the sides. The capture member 220 is preferably linked to the adjustable suture loop 218 through a lateral aperture 254 formed in the body of the capture member 220. Functionality in conjunction with the capture member 220 may be further enhanced by the provision of spikes or ridges along the outer surface thereof.

The forward first end 250 also includes a catch member 255. In accordance with a disclosed embodiment, the catch member 255 is a lateral slot positioned at the forward first end 250 of the capture member 220. While the lateral slot is shown in accordance with the disclosed embodiment, it is appreciated the lateral slot could be positioned at various locations along the capture member so long as the lateral slot is able to engage the suture loop 218 for operation in accordance with the present invention. The lateral slot is shaped and dimensioned for capture and retention of suture(s), and other surgical elements, as discussed below in greater detail. As with the prior embodiment, it is appreciated the catch member may take a variety of forms so long as it is capable of catching or snagging in accordance with the present invention.

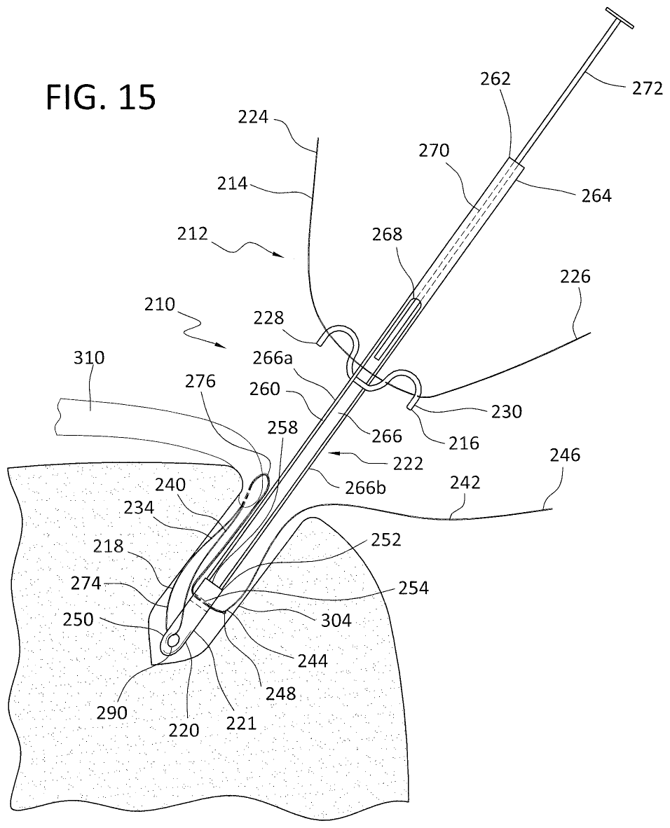

Further, as depicted in FIG. 15, in place of the catch member 255, an eyelet or opening 290 may be provided along the capture member 220, whereby the first partial suture loop 274 is threaded through the eyelet or opening 290 and then around the catch member 255 before being inserted into the bone hole 304. Additionally, the partial suture loop can 274 can be passed back through the lateral aperture 254 after passing through the tissue 310 and then captured around the end of the capture member 220 and inserted into the bone hole 304.

The rear second end 252 of the capture member 220 is shaped and dimensioned for selective coupling with the distal second end 258 of the delivery inserter 222, for example, via a threaded attachment structure.

In accordance with a preferred embodiment, the delivery inserter 222 includes an elongated body 260 with a proximal first end 262 and a distal second end 258. The proximal first end 262 is provided with a handle 264 for actuation in accordance with the present invention. The distal second end 258 is shaped and dimensioned for selective attachment to capture member 220.

The elongated body 260 further includes a slot 266 extending from the distal second end 258 to a central position 268 along the length of the elongated body 260. The slot 266 thereby defines first and second legs 266a, 266b that extend from the central position 268 along the length of the elongated body to the distal second end 258 of the elongated body 260. The slot 266 is shaped such that the all-suture anchor 212, comprised of the all-suture anchor element 216 and the associated suture strand 214, may be passed therethrough (that is, between the first and second legs 266a, 266b), in particular, the portion of the all-suture anchor 212 where the suture strand 214 is threaded through the all-suture anchoring element 216. The portion of the elongated body 260 proximal to the slot 266 is provided with an elongated cannulation 270 extending from the slot 266 to the proximal first end 262. The cannulation 270 is shaped and dimensioned to receive an actuation rod 272 that engages with the all-suture anchor 212 to insert it into a bone hole 304 in the manner discussed below in detail.

The delivery inserter 222 is operated by a surgeon whereby the surgeon captures the suture loop 218 in the catch member 255 of the capture member 220, after the suture loop 218 has been passed through the tissue 310. The capture member 220 and the suture loop 218 are then inserted in the bone hole 304 by pushing the distal second end 258 of the delivery inserter 222 into the bone hole 304. Once the capture member 220 and the captured suture loop 218 have been inserted into the bone hole 304 to the desired depth, the surgeon can push the actuation rod 272 to insert the all-suture anchor 212, in particular, the portion of the all-suture anchor 212 where the suture strand 214 is threaded through the all-suture anchor element 216, into the bone hole 304. The all-suture anchor element 216 is then folded, bent, creased, crinkled, bunched or otherwise changed in shape as it is forced into the bone hole 304 in a manner that compresses the all-suture anchor element 216 in a manner that ultimately increases the size of the all-suture anchor element 216 in a direction substantially perpendicular to the longitudinal axis of the bone hole 304 to develop an outwardly directed forced that is directed at the walls of the bone hole 304. The folding, bending, creasing, crinkling, bunching or other changes in the shape of the all-suture anchor element 216 is achieved using known techniques. While release of the catch member is described above as occurring prior to deployment of the all-suture anchoring element, it is appreciated release of the capture member 220 from the delivery inserter may take place either before, during, or after deployment of the all-suture anchoring element. As shown, and further to the discussion above, the portion of the suture loop 218 opposite the slip knot 248 is pulled then through the tissue 310. It is also appreciated the slip knot 248 or the alternative suture loop shortening mechanism (for example, Chinese finger trap structure as mentioned above) can be pulled through the tissue, if desired, to allow for this adjustment mechanism to be located on the other side of tissue 310. This creates a first partial suture loop 274 on one side of the tissue 310 and a second partial suture loop 276 on the other side of the tissue 310. The capture member 220, to which the delivery inserter 222 is attached, is then used to capture the first partial suture loop 274 for insertion into a bone hole 304.

The capture member 220 and the first partial suture loop 274 are then inserted into the bone hole 304. The size of the loop member 240 can be adjusted before or after insertion into bone hole 304 by pulling upon the tensioning suture component 242, and further tensioning of the adjustable suture loop 218 (resulting in a reduction in the size of the loop member 240) might be achieved by pulling on the tensioning suture component 242 even after anchor fixation. Pulling on tensioning suture component 242 will adjust the size of loop member 240. The all-suture anchor assembly 212 is deployed to provide fixation to bone and to secure the capture member 220 in the bone hole 304. Tissue reduction, achieved by the reduction in the size of loop member 240 by pulling on tensioning suture component 242, can be achieved before, during, and/or after deployment and fixation by the all-suture anchor assembly 212. Deployment of the anchor assembly is initiated by pressing downwardly upon the actuation rod 272 to force the all-suture anchoring element 216 into the bone hole 304 and the all-suture anchoring element 216 is tensioned causing the all-suture anchoring element 216 to fold, bend, crease, crinkle, bunch or otherwise change shape in a manner that compresses the all-suture anchoring element 216. It is appreciated deployment may occur before, after, or as the delivery inserter 222 is removed. It is also appreciated that reduction in the size of loop member 240 of the adjustable suture loop 218 to tension the tissue being repaired, may occur before, during and/or after deployment of the all-suture anchor element 216.

As the all-suture anchoring element 216 is compressed in this manner it ultimately increases in size in a direction substantially perpendicular to the longitudinal axis of the bone hole 304 (or otherwise oriented to contact side walls of the bone hole 304) and develops an outwardly directed forced that is directed at the walls of the bone hole 304. As such, and when tension is no longer being applied by the delivery inserter 222, or other delivery instrument, the all-suture anchoring element 216 exhibits an outward bias resulting in expansion of the all-suture anchoring element 216 in a direction substantially perpendicular to the longitudinal axis of the bone hole 304 (or otherwise oriented to contact side walls of the bone hole 304) into which it is positioned, resulting in frictional engagement or gripping the wall of the cancellous bone, which is referred to herein as the expanded deployed state.

The all-suture anchoring element 216 is folded or otherwise compressed to form pleats between adjacent suture aperture locations 232a-d. This pleating reduces the distance between the first suture aperture location 232a and the second suture aperture location 232b, as measured along the length of the all-suture anchoring element 216. These pleats form a bunched mass of suture material effectively increasing a diameter, or cross sectional dimension, (as measured in relation to the axis of the bone hole 304) of the all-suture anchoring element 216, which ultimately causes the all-suture anchor assembly 210 to displace cancellous bone. It should be appreciated that this displacement of bone might not occur if the all-suture anchor 212 is inserted into hard bone or a sleeve. The outward force, due to anchor deployment, will maintain the position of the all-suture anchor 212 within the bone hole or sleeve, even if there is not cancellous bone displacement. The relative increase in the cross sectional size of the all-suture anchoring element 216 in the direction substantially perpendicular to the longitudinal axis of the bone hole 304 (or in another direction to facilitate contact of the all-suture anchoring element 216 with the side walls of the bone hole 304) creates a retention force of the all-suture anchor assembly 210.

The capture member 220, the suture loop 218, the suture strand 214, and the all-suture anchoring element 216 are installed within the bone hole 304 in an elongated fashion to take advantage of a small diameter configuration, referred to herein as an undeployed state or installation state. Deployment occurs as the delivery inserter 222 is manipulated such that the capture member 220 captures and retains the first partial suture strand 274 and other surgical elements. This capture and retention are followed by positioning of the first partial suture strand 274 and the capture member 220 within a bone hole 304 for retention of the various elements to a bone mass.

As with the prior embodiment disclosed above with reference to FIGS. 1 to 9, it is appreciated a mechanical tensioning mechanism, as is well known to those skilled in the art, may be used during the deployment of the all-suture anchoring element.

It should be appreciated that the relative fit of the all-suture anchor 212 in the bone hole 304 in its deployed configuration is shown as being relatively “loose.” This is done to provide a clear view of the elements making up the present invention. In practice, it is appreciated that the suture loop 218, all-suture anchoring element 216, the capture member 220, a portion of the suture 214, and delivery inserter 222 would be tightly pressed into the bone hole 304, as any excess space would need to be taken up by the expansion of the all-suture anchor assembly in a direction substantially perpendicular to the longitudinal axis of the bone hole 304 (or otherwise oriented to contact side walls of the bone hole 304).

In accordance with an alternate embodiment, and as described in U.S. Patent Application Publication No. 2017/0290578, entitled “Modified Adjustable, Locking All-Suture Anchor Assembly And Method For Repair,” which is incorporated herein by reference, a sleeve may be used to enhance anchoring of the all-suture anchoring element 216 within the bone hole. This is particularly valuable when soft bone or bone cysts are encountered that might reduce the security achieved by the deployed all-suture anchor.

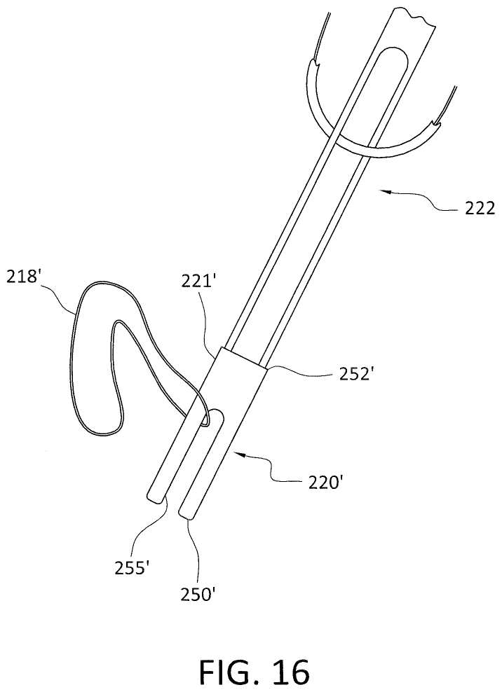

While the above disclosure provides for an adjustable suture loop element 218 passing through the capture member 220, it is appreciated other mechanisms for integrating the adjustable suture loop element may be employed. For example, and with reference to FIG. 16, the capture member 220′ is formed without the lateral aperture described above. In accordance with such an embodiment, the delivery inserter 222 is the same as described above. As to the solid capture member 220′, it includes an elongated body 221′ having a forward first end 250′ and a rear second end 252′. The forward first end 250′ also includes a catch member 255′. The catch member 255′ is a lateral slot positioned at the forward first end 250′ of the capture member 220′. As with the prior embodiment, while the lateral slot is shown in accordance with the disclosed embodiment, it is appreciated the lateral slot could be positioned at various locations along the capture member so long as the lateral slot is able to engage the suture loop 218′ (either a fixed or adjustable loop) for operation in accordance with the present invention. Also, the catch member may take a variety of forms so long as it is capable of catching or snagging in accordance with the present invention, and therefore may take various shapes and have various dimensions without departing from the spirit of the present invention.

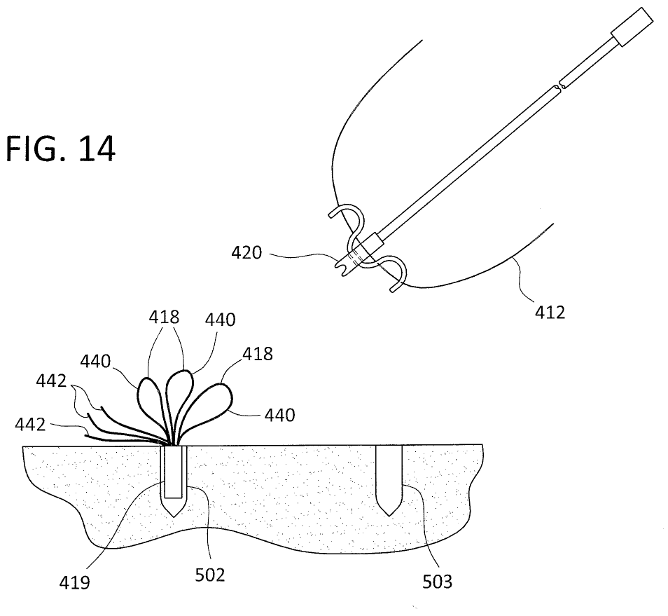

Although a single hole repair is described above, it is appreciated that more extensive repairs can comprise multiple drilled bone holes resulting in a dual row procedure. In accordance with such an embodiment, and with reference to FIG. 14, the suture loop 218 described above is replaced with one or more adjustable suture loops 418, each with a loop member 440, that attached to a single bone anchor 419. The single bone anchor 419 is inserted into a bone hole 502. It is appreciated the single bone anchor 419 may be take a variety of forms and may be secured within the bone hole 502 using any standard anchor mechanism (for example, threads, prongs, wedge, etc). One or each of the plurality of loop members 440 is passed through the tissue 510 to be repaired at one or more locations.

One at a time, the loop members 440 are then snagged by capture members 420 and the insertion procedure takes place as described above. In particular, a capture member 420 and the first of the plurality of loop members 440 are then inserted into the bone hole 503. The loop size can be adjusted before, during, and/or after insertion into the bone hole 503 by pulling upon the tensioning suture component 442 of the adjustable suture loop 418. Additional tensioning of the suture loop 418 might be achieved by pulling on the tensioning suture component 242 thereof even after anchor fixation. The all-suture anchor 412 is deployed to provide fixation to bone and to secure the capture member 420 in the bone hole 503. Deployment and fixation of the all-suture anchor 412 is achieved before, during, and/or after reduction in the size of adjustable suture loop 418. This process is repeated for each of the plurality of adjustable suture loops 418. As a result, multiple suture loops from one anchor can be captured by multiple capture members and inserted into multiple drill holes (that is, as required for dual-row rotator cuff repair).

As with the prior embodiment disclosed above with reference to FIGS. 1 to 9, with the inclusion of a solid capture member as discussed above, the methodology may be altered considering a bone hole may not be required, and the solid capture member may be used to penetrate the bone mass and form a cavity into which the all-suture anchor assembly is positioned. Also, and as with the prior embodiment disclosed above with reference to FIGS. 1 to 9, the all-suture knotless repair system described above may be employed in a variety of medical procedures.

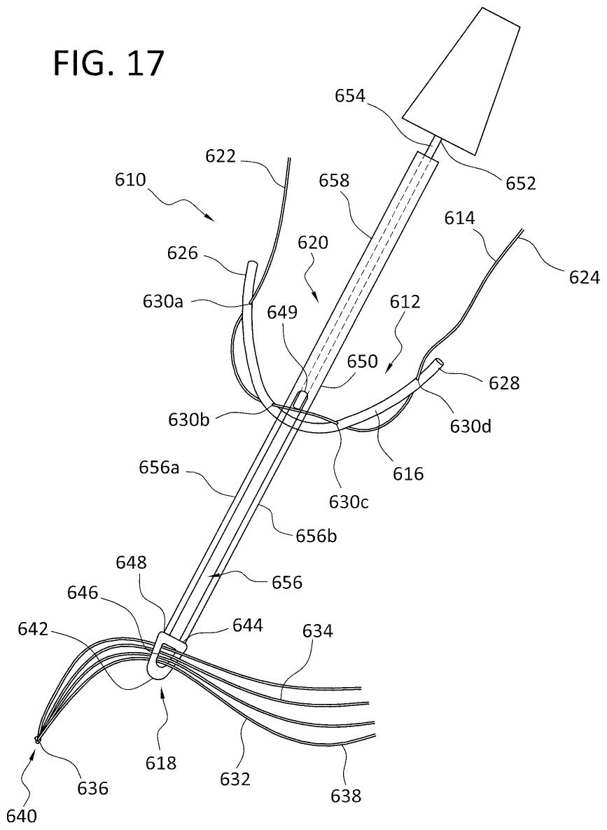

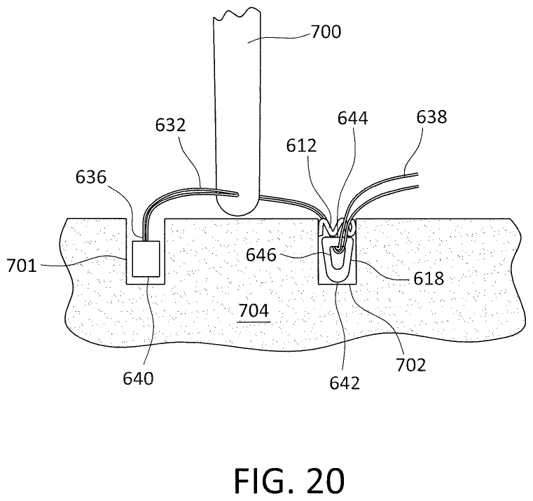

In accordance with the another embodiment, and with reference to FIGS. 17 to 20, an all-suture anchor assembly 610 and associated method are disclosed. The all-suture anchor assembly 610 includes an all-suture anchor 612 composed of a suture strand 614 and an all-suture anchoring element 616 threaded along the suture strand 614 in manner discussed below in greater detail. The all-suture anchor assembly 610 also includes an islet tip 618 attached to a delivery inserter 620. The islet tip 618 can be linked to one or a plurality of elongated sutures 632, 634. The suture(s) 632, 634 can be an independent suture or it can be attached to a separate anchor 640.

As with the prior embodiment disclosed above with reference to FIGS. 1 to 9, it should be appreciated that while a preferred all-suture anchor 612 is disclosed below in accordance with a preferred embodiment, the all-suture anchor 612 may take a variety of forms as known in the art. The suture strand 614 has a suture first end 622 and a suture second end 624. The all-suture anchoring element 616 is preferably composed solely of an enlarged piece of cylindrical suture material or a suture tape. The enlarged surface area of the all-suture anchoring element 616 allows for the passage of the suture strand 614 therethrough in a manner providing for entanglement of the all-suture anchor element 616 and the suture strand 614. The suture anchoring element 616 includes a first end 626 and a second end 628, as well as a thickness, a width and a length along a longitudinal axis.

As briefly mentioned above, the all-suture anchor assembly 610 includes the suture strand 614, which is passed through the all-suture anchoring element 616 at various locations along the length of the all-suture anchoring element 616. That is, the suture penetrates and traverses the all-suture anchoring element 616 so as to define apertures in the all-suture anchoring element 616. The intersections of the suture with the all-suture anchoring element 616 are referred to herein as suture aperture locations 630a-d and, as such, each of the suture aperture locations 630a-d is a location where the suture strand 614 passes through the thickness of the all-suture anchoring element 616. As shown in FIGS. 17 to 19, there are four suture aperture locations 630a-d. It is appreciated that as few as three suture aperture locations on a particular all-suture anchor assembly may function well. Similarly, more suture aperture locations may be provided, although it has been discovered that each additional suture aperture location increases friction against the suture thus reducing a surgeon's ability to slide the suture in relation to the all-suture anchoring element 616. While the suture aperture locations 630a-d disclosed above in accordance with a preferred embodiment are all centrally located along the all-suture anchoring element 616 so as to be oriented along the central longitudinal axis of the all-suture anchoring element 616, it is appreciated the suture aperture locations 630a-d may be varied (for example, staggered on opposite sides of the central longitudinal axis of the all-suture anchoring element 616) without departing from the spirit of the present invention. In accordance with a preferred embodiment, the all-suture anchoring element 616 may have various length and width dimensions depending upon the purpose for which it is intended.

While the suture strand 614 is disclosed above as being threaded through the all-suture anchoring element 616, it is appreciated that suture strand 614 could be passed through a central passageway defined by the all-suture anchoring element 616 where the all-suture anchoring element 616 is constructed as an elongated cylindrical member. Similar, and while the embodiment above discloses the all-suture anchoring element 616 as having separate ends, the ends of the all-suture anchoring element 616 could certainly be connected as is known in the art.

With the foregoing in mind, it is appreciated that a large variety of constructions and materials will work for the all-suture anchor assembly 612. It has been discovered that for each type of construction (i.e., braided, woven, non-woven, or knitted) there is an advantage for using a material that increases in width for every reduction in length. This advantage provides for increased diameters for a particular number of folds, pleats, crinkles or other changes in the shape of the all-suture anchor element 614. Regardless of the material chosen for use in accordance with the present invention, the material must exhibit desirable deformation and retention characteristics.

While the elongated suture(s) 632, 634 are disclosed herein in describing the present invention, it is appreciated the all-suture anchor assembly 610 of the present invention does not require the elongated suture(s) 632, 634, but may be used in conjunction with various other known medical coupling structures. Rather, the all-suture anchor assembly 610 is ‘linked to’ these sutures, and the suture(s) 632, 634 can be attached to an anchor 640 or the suture(s) 632, 634 can be a free suture(s) without attachment to an anchor 640. If a free suture(s) is used, the suture(s) would be passed through the tissue, and then passed through the islet 618, before insertion of the all-suture anchor assembly 610 into bone. If the suture(s) 632, 634 are linked to an anchor 640, then the procedure is as described herein.

The all-suture anchor assembly 610 further includes the solid (polymer or any material) islet tip 618 that is ultimately linked to the elongated suture(s) 632, 634 for the purpose of enhancing the surgeon's ability to position the elongated suture(s) 632, 634 and all-suture anchor 612 within the bone mass 704. In accordance with a preferred embodiment, the solid islet tip 618 includes a forward first end 642 and a rear second end 644. The islet tip 618 is preferably secured to the elongated suture(s) 632, 634 by passing the elongated suture(s) 632, 634 through an aperture 646 formed in the body of the islet tip 618.

The rear second end 644 of the islet tip 618 is shaped and dimensioned for selective coupling with the distal second end 648 of the delivery inserter 620, for example, via a threaded attachment structure.

In accordance with a preferred embodiment, the delivery inserter 620 includes an elongated body 650 with a proximal first end 652 and a distal second end 648. The proximal first end 652 is provided with a handle 654 for actuation in accordance with the present invention. The distal second end 648 is shaped and dimensioned for selective attachment to islet tip 618.

The elongated body 650 further includes a slot 656 extending from the distal second end 648 to a central position 649 along the length of the elongated body 650. The slot 656 thereby defines first and second legs 656a, 656b that extend from the central position 649 along the length of the elongated body 650 to the distal second end 648 of the elongated body 650. The slot 656 is shaped such that the all-suture anchor 612 may be passed therethrough (that is, between the first and second legs 656a, 656b), in particular, the portion of the all-suture anchor 612 where the suture strand 614 is threaded through the all-suture anchoring element 616. The portion of the elongated body 650 proximal to the slot 656 is provided with an elongated cannula 658 extending from the slot 656 to the proximal first end 652. The cannula 658 is shaped and dimensioned to receive an actuation rod 660 that engages with the all-suture anchor 612 to advance and insert it into a bone hole 702 in the manner discussed below in detail.

The delivery inserter 620 is operated by a surgeon whereby the surgeon captures the suture(s) 632, 634 and inserts the islet tip 618 and the elongated suture 632 in the bone hole 702 by pushing the distal second end 648 of the delivery inserter 620 into the bone hole 702. At that point, the surgeon can push the actuation rod 660 to advance and insert the all-suture anchor 612, in particular, the portion of the all-suture anchor 612 where the suture strand 614 is threaded through the all-suture anchoring element 616, into the bone hole 702.

The surgeon then actuates the delivery inserter 620 to release the islet tip 618 from the distal second end 648 of the delivery inserter 620. The all-suture anchoring element 616 is then folded, bent, creased, crinkled, bunched or otherwise changed in shape as it is forced into the bone hole 702 in a manner that compresses the all-suture anchoring element 616 in a manner that ultimately increases the size of the all-suture anchoring element 616 in a direction substantially perpendicular to the longitudinal axis of the bone hole 702 to develop an outwardly directed forced that is directed at the walls of the bone hole 702. The folding, bending, creasing, crinkling, bunching or other changes in the shape of the all-suture anchoring element 616 is achieved using known techniques. While release of the islet tip is described above as occurring prior to deployment of the all-suture anchoring element, it is appreciated release of the islet tip from the delivery inserter may take place either before, during, or after deployment of the all-suture anchoring element.

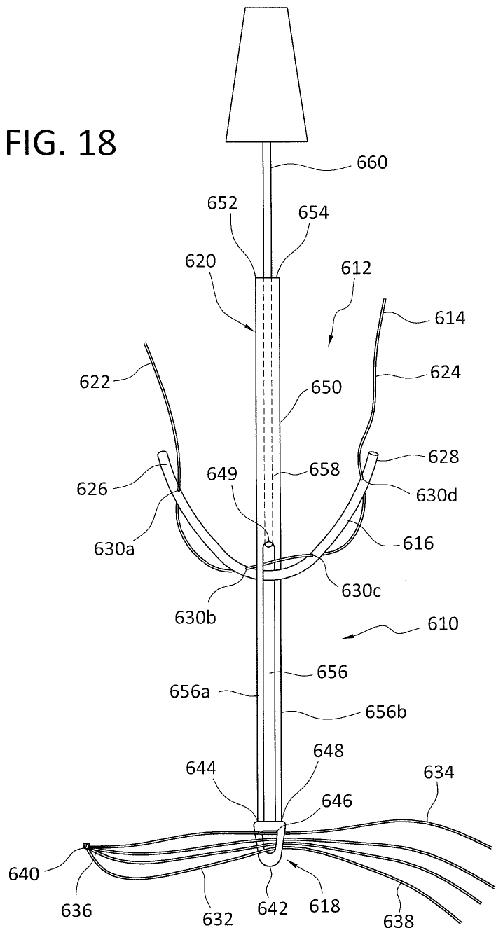

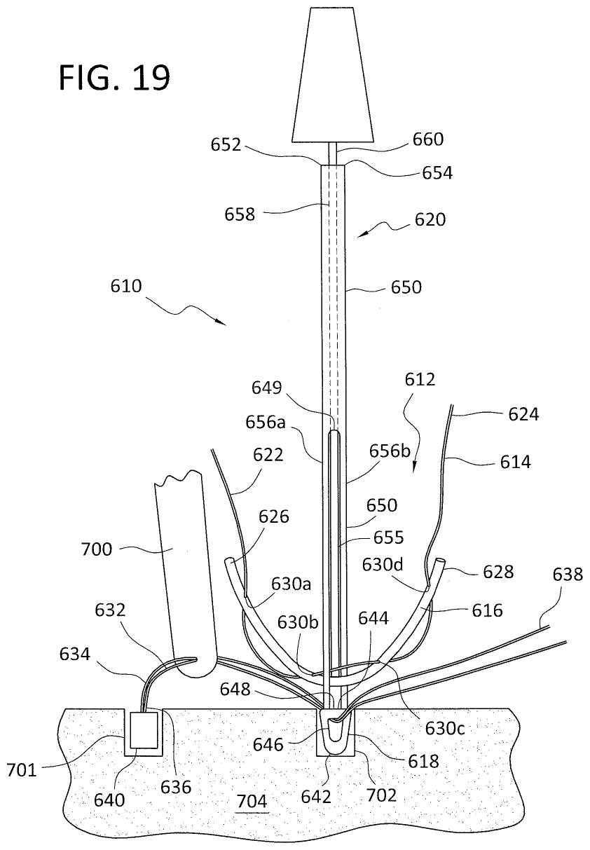

As shown with reference to FIGS. 18 and 19, and further to the discussion above, with the first end 636 of the elongated suture(s) 632, 634 secured to the bone anchor 640 and the bone anchor 640 deployed in a first bone hole 701, the second end(s) 238 of the elongated suture(s) 232, 234 is pulled through the tissue 700 and is then pulled thought the aperture 246 of the islet tip 218.

The islet tip 218 and the portions of the elongated suture(s) 232, 234 passing therethrough are then inserted into a second bone hole 702. The tension can be adjusted before, during, and/or after insertion into bone hole 702 by pulling upon the second end(s) 638 of the elongated suture 632, and further tensioning of the suture 632 might be achieved by pulling on the second end(s) 638 thereof even after anchor fixation. After the desired tensioning is achieved, the all-suture anchor 612 is deployed to provide fixation to bone and to secure the islet tip 618 in the bone hole 702.

Deployment of the all-suture anchor 612 is initiated as the all-suture anchoring element 616 is tensioned causing the all-suture anchoring element 616 to fold, bend, crease, crinkle, bunch or otherwise change shape in a manner that compresses the all-suture anchoring element 616. It is appreciated that deployment may occur before, after, or as the delivery inserter 620 is removed. As the all-suture anchoring element 616 is compressed in this manner it ultimately increases in size in a direction substantially perpendicular to the longitudinal axis of the bone hole 702 (or otherwise oriented to contact side walls of the bone hole 702) and develops an outwardly directed forced that is directed at the walls of the bone hole 702. As such, and when tension is no longer being applied by the delivery inserter 620, or other delivery instrument, the all-suture anchoring element 616 exhibits an outward bias resulting in expansion of the all-suture anchoring element 616 in a direction substantially perpendicular to the longitudinal axis of the bone hole 702 (or otherwise oriented to contact side walls of the bone hole 702) into which it is positioned, resulting in frictional engagement or gripping the wall of the cancellous bone, which is referred to herein as the expanded deployed state.

The all-suture anchoring element 616 is folded or otherwise compressed to form pleats between adjacent suture aperture locations 630a-d. This pleating reduces the distance between the first suture aperture location and the second suture aperture location, as measured along the length of the all-suture anchoring element 616. These pleats form a bunched mass of suture material effectively increasing a diameter, or cross sectional dimension, (as measured in relation to the axis of the bone hole 702) of the all-suture anchoring element 616, which ultimately causes the all-suture anchor assembly 610 to displace cancellous bone. The relative increase in the cross sectional size of the all-suture anchoring element 616 in the direction substantially perpendicular to the longitudinal axis of the bone hole 702 (or in another direction to facilitate contact of the all-suture anchoring element 616 with the side walls of the bone hole 702) creates a retention force of the all-suture anchor assembly 610.

The islet tip 618, the elongated suture(s) 632, 634, the suture strand 614, and the all-suture anchoring element 616 are installed within the bone hole 702 in an elongated fashion to take advantage of a small diameter configuration, referred to herein as an undeployed state or installation state. Deployment occurs as the delivery inserter 620 is manipulated such that the islet tip 618 captures and temporarily retains the first partial suture strand and other surgical elements. This capture and temporary retention are followed by positioning of the first partial suture strand and the islet tip 618 within a bone hole 704 for retention of the various elements to a bone mass.

It is appreciated a mechanical tensioning mechanism, as is well known to those skilled in the art, may be used during the deployment of the all-suture anchoring element. Such mechanical tensioning mechanisms pull or ratchet the suture while the delivery inserter 620 holds the all-suture anchoring element in place. Mechanical tensioning, of this nature, may be preferable as this can more tightly ‘fold’ or ‘bunch’ the all-suture anchoring element, thereby increasing the created tension, that is, the outward force of the all-suture anchoring element, which is relative to the longitudinal axis of the bone hole and toward the walls of the bone hole, resulting from increased compression of the all-suture anchoring element. The changed shape of the all-suture anchoring element provides security within the bone, for example, below the cortical layer.

It should be appreciated that the relative fit of the all-suture anchor 612 in the bone hole 702 in its deployed configuration is shown as being relatively “loose.” This is done to provide a clear view of the elements making up the present invention. In practice, it is appreciated that the suture, all-suture anchoring element 616, the islet tip 618, a portion of the suture 612, and delivery inserter 620 would be tightly pressed into the bone hole 702, as any excess space would need to be taken up by the expansion of the all-suture anchor 612 in a direction substantially perpendicular to the longitudinal axis of the bone hole 702 (or otherwise oriented to contact side walls of the bone hole 702).

In accordance with an alternate embodiment, and as described in U.S. Patent Application Publication No. 2017/0290578, entitled “MODIFIED ADJUSTABLE, LOCKING ALL-SUTURE ANCHOR ASSEMBLY AND METHOD FOR REPAIR,” which is incorporated herein by reference, a sleeve may be used to enhance anchoring of the all-suture anchoring element 616 within the bone hole.

With the inclusion of a solid islet tip as discussed above, the methodology may be altered considering a bone hole may not be required, and the islet tip may be used to penetrate the bone mass and form a cavity into which the all-suture anchor is positioned. In accordance with such an embodiment, and with the delivery inserter directly attached to the islet tip, the forward first end of the islet tip is impacted directly into bone mass, with the remainder of the islet tip following and being inserted into the bone mass. Thereafter, the all-suture anchoring element and the anchor suture strand are forced into the hole created by the islet tip, and the all-suture anchoring element is folded, bent, creased, crinkled, bunched or otherwise changed in shape in a manner that compresses the all-suture anchoring element as discussed above. Other than the inclusion of the solid islet tip and the direct impacting into the bone mass, the methods for use disclosed herein remain the same.

The all-suture knotless repair systems described above may be employed in a variety of medical procedures. In many situations throughout the discussion above, the terminology relating to the secure attachment of soft tissue to bone mass has been used. Such terminology refers to the attachment or reattachment of tissue to a bone mass by securely binding the tissue to the bone mass utilizing the novel knotless suture anchor assembly. The suture element can be made up of a known suture material, or it can be made of polymer materials, or can be formed of bioabsorbable/biocomposite material such as a polylactide polymer.

While the preferred embodiments have been shown and described, it will be understood that there is no intent to limit the invention by such disclosure, but rather, is intended to cover all modifications and alternate constructions falling within the spirit and scope of the invention.

1. An all-suture anchor assembly, comprising:
an all-suture anchor composed of at least one suture strand and an all-suture anchoring element threaded along the suture strand; and
a capture member integrated with the all-suture anchor for controlled capture of various elements, the capture member includes an elongated body having a forward first end, a rear second end, a catch member, and a lateral aperture with a suture passing therethrough.

2. The all-suture anchor assembly according claim 1, wherein the at least one suture strand has a suture first end and a suture second end which are secured together at an intersection.

3. The all-suture anchor assembly according claim 1, wherein the catch member is a lateral slot positioned at the forward first end of the capture member.

4. The all-suture anchor assembly according claim 1, wherein the rear second end of the capture member is shaped and dimensioned for selective coupling with a delivery inserter.

5. The all-suture anchor assembly according claim 1, further including a delivery inserter having an elongated body with a proximal first end provided with a handle for actuation and a distal second end shaped and dimensioned for selective attachment to capture member.

6. The all-suture anchor assembly according claim 5, wherein the elongated body includes a slot extending from the distal second end to a central position along the length of the elongated body, wherein the slot is shaped such that the all-suture anchor may be passed therethrough.

7. The all-suture anchor assembly according claim 6, wherein a portion of the elongated body proximal to the slot is provided with an elongated cannulation extending from the slot to the proximal first end, and the cannulation is shaped and dimensioned to receive an actuation rod that engages with the all-suture anchor.

8. The all-suture anchor assembly according claim 1, wherein the at least one suture strand is an adjustable suture strand.

9. The all-suture anchor assembly according claim 1, wherein the at least one suture strand is threaded through the all-suture anchoring element.

10. The all-suture anchor assembly according claim 1, further including a suture loop.

11. An all-suture anchor assembly, comprising:
an all-suture anchor composed of at least one suture strand and an all-suture anchoring element threaded along the suture strand; and
a capture member or islet tip; and
a delivery inserter including an elongated body with a proximal first end and the distal second end shaped and dimensioned for selective attachment to the capture member or the islet tip, the elongated body includes a slot extending from the distal second end to a central position along the length of the elongated body, wherein the slot is shaped such that the all-suture anchor may be passed therethrough, and a portion of the elongated body proximal to the slot is provided with an elongated cannulation extending from the slot to the proximal first end, and wherein the cannulation is shaped and dimensioned to receive an actuation rod that engages with the all-suture anchor.

12. The all-suture anchor assembly according claim 11, wherein the capture member is integrated with the all-suture anchor.

13. The all-suture anchor assembly according claim 11, wherein the at least one suture strand is threaded through the all-suture anchoring element.

14. The all-suture anchor assembly according claim 11, further including a suture loop.

15. The all-suture anchor assembly according claim 11, wherein the capture member or islet tip is a capture member including a catch member.

16. The all-suture anchor assembly according claim 15, wherein the catch member is a lateral slot positioned at the forward first end of the capture member.

17. The all-suture anchor assembly according claim 11, wherein the capture member or islet tip is an islet tip islet tip having a body with an aperture formed therein.

18. The all-suture anchor assembly according claim 17, further including a plurality of elongated sutures passing through the aperture formed in the islet tip.

19. The all-suture anchor assembly according claim 18, further including a bone anchor secured to the plurality of elongated sutures.

20. The all-suture anchor assembly according claim 11, wherein the slot defines first and second legs.
